(19) United States
(12) Patent Application Publication
(10) Pub. No.: US 2012/0213661 A1
(43) Pub. Date: Aug. 23, 2012

(54) WARM-UP SYSTEM FOR CATOX DECONTAMINATION SYSTEM

(75) Inventor: DAVID LOEFFELHOLZ, LONG BEACH, CA (US)

(73) Assignee: HONEYWELL INTERNATIONAL INC., MORRISTOWN, NJ (US)

(21) Appl. No.: 13/032,976

(22) Filed: Feb. 23, 2011

(51) Int. Cl. A61L 9/00; B01D 53/86

(52) U.S. Cl. 422/4; 422/159; 422/168; 422/169

(57) ABSTRACT

A decontamination system useful for treating articles that have been exposed to nuclear, chemical or biological (NBC) agents may comprise a decontamination unit, a catalytic oxidation (CATOX) reactor connected to decontaminate off gases from the decontamination unit and a selector valve The selector valve may be configured to allow output gases from the CATOX reactor to pass to an exhaust during steady-state operation of the CATOX reactor and to allow said output gases to re-enter the CATOX reactor during warm-up of the CATOX reactor. Warm-up of the CATOX reactor may be quickly achieved as a result of recycling the output gases through the preheater during warm-up.

### GOVERNMENT RIGHTS

This invention was made with Government support under ALS CATOX Program Contract N00178-05-D-42 awarded by US Army-ECBC. The Government has certain rights in this invention.

### BACKGROUND OF THE INVENTION

The present invention generally relates to catalytic-oxidation (CATOX) converters and more particularly to CATOX converters used to treat off gases from decontamination units.

In some military and/or civil defense operations it may become necessary to decontaminate objects that may have been exposed to nuclear, biological or chemical (NBC) contamination. Such NBC decontamination may be performed in an isolatable chamber such as a glove-box. NBC decontamination activity may produce off-gases and these off-gases may also carry some NBC contamination. In some instances, the NBC contaminated off gases may be treated with filtration systems and then released to the atmosphere. In other case, it has been found desirable to treat the off gases with a CATOX system before releasing the gases to the atmosphere.

A CATOX reactor may operate effectively only after the temperature of catalytic material reaches a so-called “light-off” temperature. Thus a CATOX reactor may require warm-up before becoming fully operational. Typically, warm-up is achieved by introducing heat from a preheater into the CATOX reactor. During a warm-up period, it is usual practice to increase the preheater energy output rate beyond its normal or steady-state energy output rate. Consequently, such a preheater may be sized to produce energy output rates designed to provide warm-up of the CATOX reactor. In other words, a prior art preheater that performs warm-up may be larger than such a preheater would be if it were only required to deliver energy at a rate needed for steady-state operation of the CATOX system.

A system used in the context of NBC decontamination may be operated only intermittently. But, when there is a need for such decontamination the system must be able to quickly become operational. Thus prior art heaters used in NBC decontamination systems may be sized to produce warm-up heat at a high rate so that warm-up may be performed quickly.

As can be seen, there is a need for a CATOX system in which warm-up may be performed quickly with a relatively small-sized preheater. More particularly there is a need for such a system in which the preheater may be sized to be no larger than that needed for steady-state operation of the CATOX system.

### SUMMARY OF THE INVENTION

In one aspect of the present invention, a decontamination system may comprise: a decontamination unit; a catalytic oxidation (CATOX) reactor connected to decontaminate off gases from the decontamination unit; and a selector valve configured to allow output gases from the CATOX reactor to pass to an exhaust during steady-state operation of the CATOX reactor and to allow said output gases to re-enter the CATOX reactor during warm-up of the CATOX reactor.

In another aspect of the present invention, a CATOX system having a steady-state mode of operation and a warm-up mode of operation may comprise: a CATOX reactor; a preheater positioned to preheat input gas to the reactor; a selector valve configured to allow all output gases from the CATOX reactor to pass to an exhaust during the steady-state operation and to allow all of said output gases to re-enter the preheater and the CATOX reactor during the warm-up mode of operation.

In still another aspect of the invention, a method for decontaminating off gases from a decontamination unit may comprise the steps of: heating input gas for a CATOX reactor; passing the heated input gas through the CATOX reactor to warm-up the reactor; redirecting output gas from the CATOX reactor into the preheater and the reactor to continue the warm-up until the CATOX reactor reaches a desired steady-state operating temperature; directing the output gases from the CATOX reactor to an exhaust after the CATOX reactor reaches the steady-state operating temperature; allowing the off gases from the decontamination unit to enter the CATOX reactor only after the temperature of the reactor is at the steady-state operating temperature so that the off gases are decontaminated by the CATOX reactor.

These and other features, aspects and advantages of the present invention will become better understood with reference to the following drawings, description and claims.

### BRIEF DESCRIPTION OF THE DRAWINGS

FIG. 1 is a block diagram of a decontamination system in accordance with an embodiment of the invention;

FIG. 2 is a graph showing a relationship between warm-up time and heat energy needed to produce warm-up of the decontamination system of FIG. 1 in accordance with an embodiment of the invention;

FIG. 3 is a block diagram of another embodiment of a decontamination system in accordance with the invention;

FIG. 4 is a graph showing a relationship between warm-up time and heat energy needed to produce warm-up of the decontamination system of FIG. 3 in accordance with an embodiment of the invention; and

FIG. 5 is a flow chart of a method for performing decontamination in accordance with an embodiment of the invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Embodiments of the present invention provide a CATOX system in which, during warm-up of a CATOX reactor, output gas from an output of the CATOX reactor may be re-cycled into a preheater.

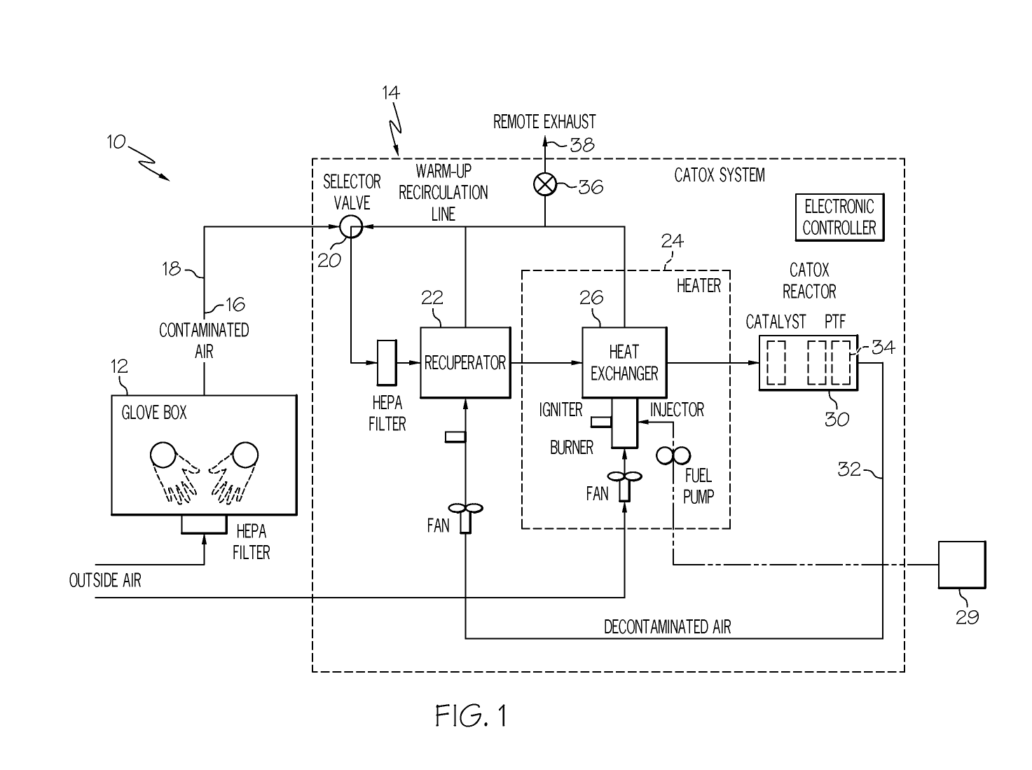

Referring now to FIG. 1, a decontamination system 10 is illustrated in block diagram format. In an exemplary embodiment of the invention, the decontamination system 10 may be employed to perform decontamination of articles which may have been exposed to nuclear, biological or chemical (NBC) agents. The decontamination system 10 may comprise a decontamination unit such as a glove box 12 and a CATOX system 14 for treating off-gases from the glove box 12. The system 10 may be housed in a vehicle (not shown) and may be employed as a mobile NBC decontamination system.

In steady-state operation, a contaminated article (not shown) may be placed in the glove box 12 and then treated with various decontamination materials (not shown). During such treatment, off-gases may be produced in the glove box 12. These off-gases may be vented out of the glove box 12 by introducing outside air into the glove box and exhausting contaminated air 16 out of the glove box 12 through a contaminated air duct 18. The contaminated air 16 may pass through a selector valve 20 and then through a recuperator 22. The contaminated air 16 may emerge from the recuperator 22 at a first elevated temperature and then pass into a preheater 24 which may raise the temperature of the contaminated air 16 to a second higher temperature. The preheater 24 may include a heat exchanger 26 and a fuel-fired burner 28. In an exemplary embodiment, the burner 28 may receive fuel from a main fuel supply 29 of a vehicle in which the system 10 may be carried. Heated contaminated air may emerge from the preheater 24 and enter a CATOX reactor 30. In an exemplary embodiment, the CATOX reactor 30 may have a monolithic design with an integrated post treatment filter (PTF) 34. Output gas 32 may emerge from the CATOX reactor 30, pass through the recuperator 22 to exchange heat with the incoming stream and then pass out of the CATOX system 14 through an exhaust 38.

An exemplary decontamination system 10 may have physical features such that, in steady-state operation, about 4 kilowatts (kW) may be continuously delivered by the preheater 24 in order to maintain an operating temperature of about 545° F. in the CATOX reactor 30 when ambient temperature is about 50° F.

When the decontamination system 10 is employed as a mobile or vehicular-mounted system for NBC decontamination, it may be operated intermittently. Consequently, there may arise numerous circumstances in which the CATOX system 14 may need to be warmed up from ambient temperature, i.e., about 50° F., to steady-state operating temperature, i.e., about 545° F. To achieve such warm-up, energy must be introduced into the CATOX system 14. In the exemplary embodiment of the CATOX system 14 described above, such warm-up energy may be about 2 kilowatt hours (kWh).

In the context of intermittent use of the decontamination system 10, it may be desirable to achieve warm-up quickly. Rapid warm-up may be achieved with a preheater that may be operated at an increased heat output rate during warm-up. However, a preheater that is capable of operating at an increased output for warm-up may be larger and more costly than a preheater that may only be required to produce energy output consistent with steady-state conditions.

In accordance with the present invention, desirably rapid warm-up may be achieved without increasing energy output of the preheater 24 beyond its steady-state output rate (e.g. about 4 kW as described above). This may be accomplished by configuring flow paths of gases through the CATOX system 14 into a temporary warm-up configuration. During warm-up, the selector valve 20 may be set so that the output gas 32 from the CATOX reactor 30 may be redirected or recycled into the preheater 24 and the CATOX reactor 30. A valve 36 may be closed so that the output gas 32 does not exit an exhaust 38 of the decontamination system 10. Thus, energy introduced by the preheater 24 may remain within the CATOX system 14. In other words, no energy may be lost through the exhaust 38 during warm-up.

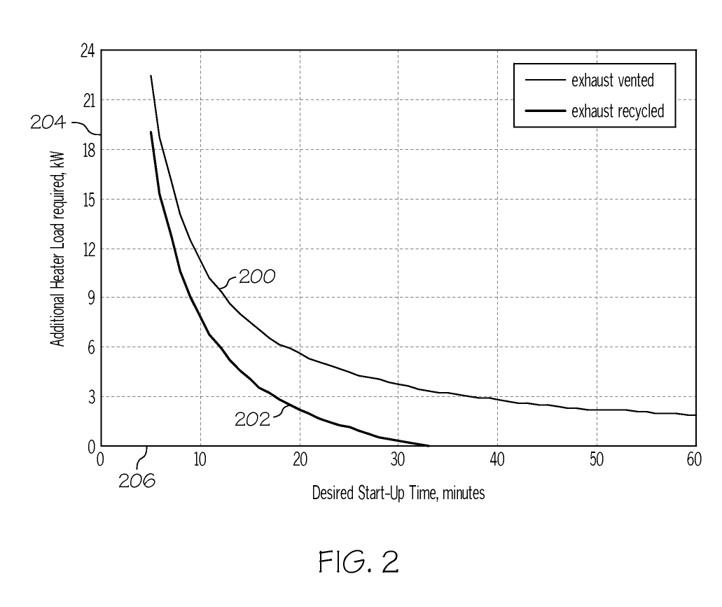

Referring now to FIG. 2, it may be seen that the above described recycling process may produce a desirable decrease in warm-up time as compared to conventional CATOX systems. FIG. 2 has graph curves 200 and 202 which illustrate a relationship between a desired warm-up time and a need for additional warm-up energy for an exemplary CATOX system 14. A vertical axis 204 shows an amount of excess energy needed beyond that required for steady-state operation. A horizontal axis 206 shows an amount of time needed to achieve a desired steady-state operating temperature. The graph curves 200 and 202 may represent a relationship expressed by the following equation:

$$Q ≅ \frac{1}{Δ t} {\sum }_{n} {( mCp [ {T}_{ss} - {T}_{init} ] )}_{n} + ( 1 - x ) {Q}_{ss}$$

Where

Q=is the heat applied to the system, in kW or similar units

mCp=is the thermal capacity of any component n in the system

Tss=average steady-state operating temperature

Tinit=temperature prior to warm up

x=fraction of process gas recirculated

Qss=is the steady state heat applied to the system, in kW or similar units

Δt=time to achieve warm-up

The curve 200 illustrates that when the output gas 32 is allowed to exit the exhaust 38, an additional warm-up energy of about 3 kW may be needed to achieve warm-up within about 40 minutes. Curve 202 illustrates that when the output gas 32 is recycled through the preheater 24 and the CATOX reactor 30, warm-up may be achieved within 30 about minutes without a need for any energy in excess of that required to sustain a steady-state when the output gas 32 exits the system at the exhaust 38.

If a warm-up time less than 40 minutes is desired, the preheater 24 must be designed and operated to provide some excess heat beyond its steady-state output. It may be seen however, that for any particular desired warm-up time, there is less excess heat output needed from the preheater 24 when the gas flow 32 is recycled through the preheater 24 and the CATOX reactor 30. Warm-up may be achievable within about 10 minutes when the preheater 24 is configured to produce energy a rate of about 4 kilowatts (kW) in the steady state-mode of operation and a rate of about 11 kW in the warm-up mode of operation, but the same warm-up time could be achieved with a preheater 24 configuration producing only about 8 kW in warm-up while recycling the gas flow 32.

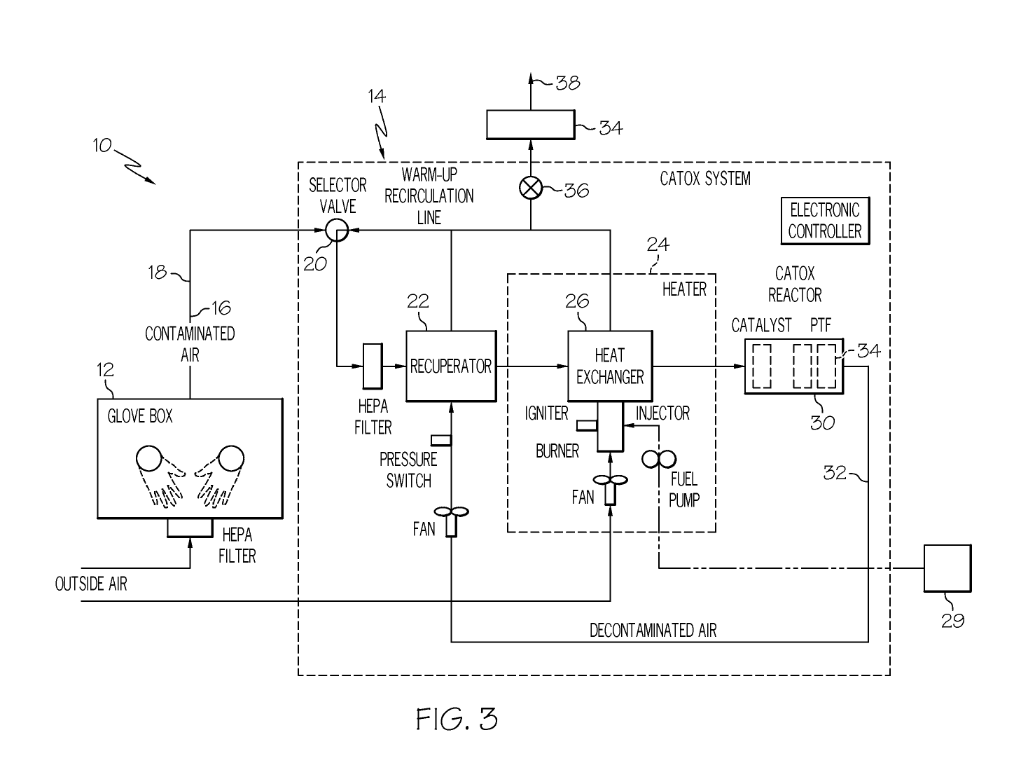

Referring now to FIG. 3, there is illustrated another exemplary embodiment of a decontamination system in accordance with the present invention. In FIG. 3, the PTF 34 may not be integral with the CATOX reactor 30. Instead, the PTF 34 may be positioned near the exhaust 38. In this position, the PTF 34 may not need to be heated during warm-up of the CATOX system 14. Thus, less energy may be required to produce warm-up.

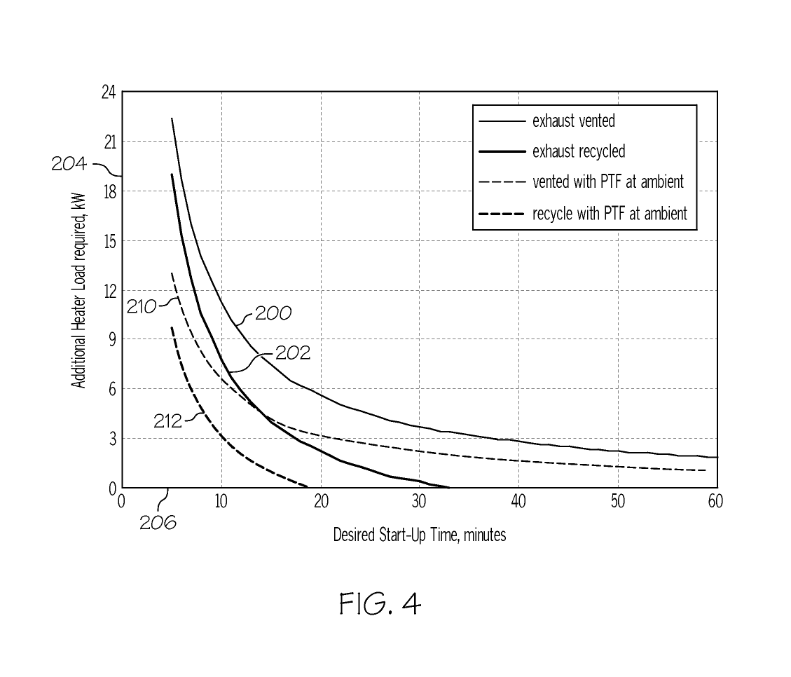

Referring now to FIG. 4, the graph curves 200 and 202 and the axes 204 and 206 illustrate the same relationships that were illustrated in FIG. 2. Graph curves 210 and 212 illustrate that when the PTF 34 is removed from the CATOX reactor 30 and is not heated during warm-up, the warm-up time is further reduced as compared to warm-up time illustrated in FIG. 2. Additionally, it can be seen that removing the PTF 34 from the CATOX reactor 30 may allow for a smaller amount of excess warm-up energy for any desired warm-up time.

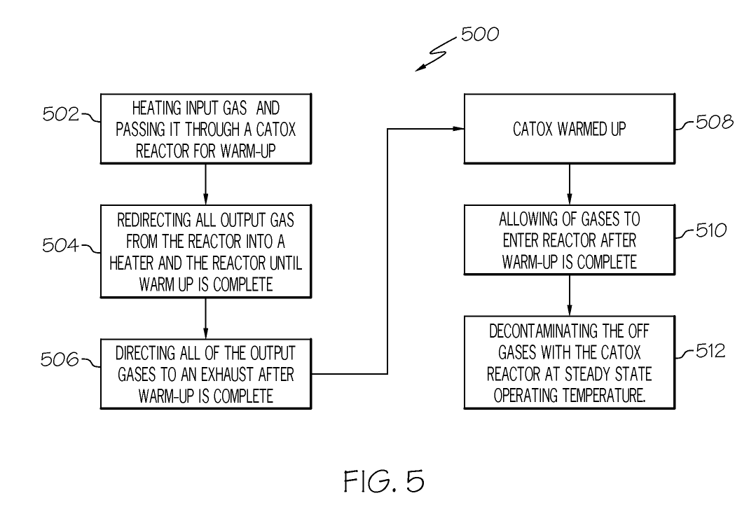

Referring now to FIG. 5, a flow chart 500 may illustrate an exemplary method which may be employed to decontaminate off gases from a decontamination unit. In a step 502, input gas for a CATOX reactor may be heated and passed through the CATOX reactor to warm-up the reactor (e.g., the preheater 24 may heat gas passing to the CATOX reactor 30). In a step 504, all output gas from the CATOX reactor may be redirected into the preheater and the reactor to continue the warm-up until the CATOX reactor reaches a desired steady-state operating temperature (e.g., all of the output gas 32 may be directed through the valve 20 to enter the preheater 24 and the CATOX reactor 30). In a step 506, all of the output gases from the CATOX reactor may be directed to an exhaust after the CATOX reactor reaches the steady-state operating temperature, (e.g., the valve 20 and the valve 36 may be positioned to direct all of the output gas 32 to the exhaust 38). In a step 508, the CATOX reactor may be determined to be warmed up. In a step 510, the off-gases from the decontamination unit may be allowed to enter the CATOX reactor only after the temperature of the reactor is at the steady-state operating temperature so that the off-gases are decontaminated by the CATOX reactor (e.g., the valve 20 may be positioned to direct the off-gases 16 into the preheater 24 and the CATOX reactor 30). In step 512, the CATOX at operating temperature may decontaminate the heated output gases.

It should be understood, of course, that the foregoing relates to exemplary embodiments of the invention and that modifications may be made without departing from the spirit and scope of the invention as set forth in the following claims.

We claim:

1. A decontamination system comprising:
a decontamination unit;
a catalytic oxidation (CATOX) reactor connected to decontaminate off-gases from the decontamination unit; and
a selector valve configured to allow output gases from the CATOX reactor to pass to an exhaust during steady-state operation of the CATOX reactor and to allow said output gases to re-enter the CATOX reactor during warm-up of the CATOX reactor.

2. The decontamination system of claim 1 further comprising a preheater configured to heat input gases for the CATOX reactor, wherein said input gases are recycled CATOX output gases re-entering the preheater during warm-up of the CATOX reactor.

3. The decontamination system of claim 2:
wherein the decontamination system is positioned in or on a vehicle; and
wherein the preheater is configured to produce energy from a main fuel supply of the vehicle.

4. The decontamination system of claim 2 wherein the preheater is configured to produce energy at a steady-state rate during both steady-state operation and warm-up of the CATOX reactor.

5. The decontamination system of claim 2:
wherein the CATOX reactor has physical features which require warm-up energy of at least about 2 kilowatt hours (kWh) to achieve warm-up from about 50° F. to about 545° F.;
wherein warm-up is achieved within about 30 minutes; and
wherein the preheater is configured to produce energy at a rate of about 4 kilowatts (kW).

6. The decontamination system of claim 1 further comprising a recuperator configured to heat input gases for the CATOX reactor, wherein said output gases re-enter the recuperator during warm-up of the CATOX reactor.

7. The decontamination system of claim 1 wherein the decontamination unit is a glove box.

8. The decontamination system of claim 1 further comprising a post treatment filter (PTF) and wherein none of the output gases pass through the PTF during warm-up.

9. A CATOX system having a steady-state mode of operation and a warm-up mode of operation comprising:
a CATOX reactor;
a preheater positioned to preheat input gas to the reactor;
a selector valve configured to allow all output gases from the CATOX reactor to pass to an exhaust during the steady-state operation and to allow all of said output gases to re-enter the preheater and the CATOX reactor during the warm-up mode of operation.

10. The CATOX system of claim 9 wherein the preheater is configured to produce heat at a steady-state rate during both the steady-state mode of operation and the warm-up mode of operation.

11. The CATOX system of claim 10 configured such that
$$Q ≅ \frac{1}{Δ t} {\sum }_{n} {( mCp [ {T}_{ss} - {T}_{init} ] )}_{n} + ( 1 - x ) {Q}_{ss}$$
wherein:
Q=is the heat applied to the system, in kW or similar units;
mCp=is the thermal capacity of any component n in the system;
Tss=average steady-state operating temperature;
Tinit=temperature prior to warm up;
x=fraction of process gas recirculated;
Qss=is the steady state heat applied to the system, in kW or similar units'
Δt=time to achieve warm-up; and
wherein x=0.

12. The CATOX system of claim 9:
wherein the CATOX system has physical features which require warm-up energy of at least about 2 kilowatt hours (kWh) to achieve warm-up from about 50° F. to about 545° F.;
wherein warm-up is achievable within about 10 minutes; and
wherein the preheater is configured to produce energy a rate of about 4 kilowatts (kW) in the steady state-mode of operation and a rate of about 11 kW in the warm-up mode of operation.

13. The CATOX system of claim 9 further comprising a post treatment filter (PTF) and wherein the PTF is positioned remotely from the CATOX reactor and none of the output gases pass through the PTF during warm-up.

14. The CATOX system of claim 13 further comprising a recuperator positioned between the CATOX reactor and the PTF.

15. A method for decontaminating off gases from a decontamination unit comprising the steps of:
heating input gas for a CATOX reactor;
passing the heated input gas through the CATOX reactor to warm-up of the reactor;
redirecting output gas from the CATOX reactor into the preheater and the reactor to continue the warm-up until the CATOX reactor reaches a desired steady-state operating temperature;
directing the output gases from the CATOX reactor to an exhaust after the CATOX reactor reaches the steady-state operating temperature;
allowing the off gases from the decontamination unit to enter the CATOX reactor only after the temperature of the reactor is at the steady-state operating temperature so that the off gases are decontaminated by the CATOX reactor.

16. The method of claim 15 further comprising the step of:
maintaining the steady-state operating temperature of the CATOX reactor by producing energy with the preheater at a steady-state rate of energy production, and
wherein the step of heating input gas for a CATOX reactor to warm-up the CATOX reactor is performed by producing energy at the same rate as the steady-state rate.

17. The method of claim 16 wherein the CATOX system is configured such that
$$Q ≅ \frac{1}{Δ t} {\sum }_{n} {( mCp [ {T}_{ss} - {T}_{inst} ] )}_{n} + ( 1 + x ) {Q}_{ss}$$
wherein:
Q=is the heat applied to the system, in kW or similar units;
mCp=is the thermal capacity of any component n in the system;
Tss=average steady-state operating temperature;
Tinit=temperature prior to warm up;
x=fraction of process gas recirculated;
Qss=is the steady state heat applied to the system, in kW or similar units'
Δt=time to achieve warm-up; and
wherein x=0.

18. The method of claim 15 wherein the CATOX system has physical features which require warm-up energy of at least about 2 kilowatt hours (kWh) to achieve warm-up from about 50° F. to about 545° F.;
wherein warm-up is achieved within about 10 minutes; and
wherein the preheater is configured to produce energy a rate of about 4 kilowatts (kW) in the steady state-mode of operation and a rate of about 11 kW in the warm-up mode of operation.

19. The method of claim 15 further comprising the step of passing the output gases through a post treatment filter (PTF) after cooling the output gases.

20. The method of claim 19 wherein the output gases are cooled by passing them through a recuperator.
